(19) United States
(12) Patent Application Publication
(10) Pub. No.: US 2020/0188610 A1
(43) Pub. Date: Jun. 18, 2020

(54) MEDICAL TOOL HAVING NEEDLE HUB

(71) Applicants: Joseph Fretta, Red Bank, NJ (US); Oneil M. Melbourne, Paterson, NJ (US)

(72) Inventors: Joseph Fretta, Red Bank, NJ (US); Oneil M. Melbourne, Paterson, NJ (US)

(21) Appl. No.: 16/718,696

(22) Filed: Dec. 18, 2019

Related U.S. Application Data
(60) Provisional application No. 62/781,225, filed on Dec. 18, 2018.

(51) Int. Cl. A61M 5/32; A61M 5/315

(52) U.S. Cl. CPC A61M 5/3293; A61M 5/3295; A61M 5/31591; A61M 2202/0007; A61M 2210/06; A61M 2210/04; A61M 5/3202

(57) ABSTRACT

A medical device for the intracellular delivery of a therapeutic formulation to a predetermined site within a patient is disclosed. The medical device comprising a needle hub assembly such that the needle hub assembly is attached to a distal end of a syringe, the needle hub assembly comprising a hollow body; a platform; and a needle hub, wherein the needle hub comprising a plurality of needles disposed on the platform arranged in a predefined needle array, wherein the plurality of needles are injected into the predetermined site within the patients' skin and/or scalp for delivering the therapeutic formulation.

### CROSS-REFERENCE TO RELATED APPLICATION

This application claims the benefit of U.S. Provisional Application Ser. No. 62/781,225 filed Dec. 18, 2018, entitled “Medical Tool Having Needle Hub”, which is incorporated herein by reference in its entirety.

### BACKGROUND

### Field of Invention

Embodiments disclosed herein relate, in general, to a medical device and, more particularly, to a medical device having a multi-needle assembly.

### Description of Related Art

Changes in environment have adverse effect on human health. The polluted air we breathe or exposure to UV radiations put humans at a higher risk of health issues such as, damage or loss of skin/tissues. Further, these health issues can alter tissue structures and/or functions which may further result in skin laxity, tightness, hair loss, etc. Several available medical tools facilitate delivery of aesthetic, and/or therapeutic compounds or formulations directly into the layers of the skin to overcome such tissue/skin conditions. However, such medical tools are not effective to overcome and/or reduce the tissue/skin disorders. For example, conventional micro needling treatment applies a patient's own plasma to the skin and then performs the micro needling to the skin, for example, facial skin, to have the serum penetrate the skin. In addition, these conventional devices attempt to infuse target areas of the skin such as, face and scalp, with concentrated stem cells, collagen and growth hormone extracted from the patient's own blood. However, these micro needling causes multiple facial punctures to stimulate plasma growth by damaging the superficial layers of the skin. These micro needling tools and its usage for treatments of skin/tissue disorders are not effective because the punctures in the superficial layers of the skin cause bleeding out to the surface. In addition, these micro needling tools do not penetrate plasma into the dermis and epidermis of a patient.

Moreover, conventional needle platform tools have a limited number of needles, for example, five or seven needles on the head to inject mesotherapy medications in order to dissolve fat. In addition, the platform space between needles are too distant, which is not beneficial for injecting plasma for skin and scalp injections. These needles are too long and thick (0.5 inches long & 27 gauge thick) to be used effectively for infiltrating epidermis of the skin with PRP (platelet rich plasma), hyaluronic acid and/or anti-inflammatory steroid.

Accordingly, it is desired to provide an improved medical tool for treatment of skin/tissue disorders and hair loss.

### SUMMARY

Embodiments of the present invention are directed to a medical device for the intracellular delivery of a therapeutic formulation to a predetermined site within a patient, according to an embodiment of the present invention. The medical device comprising a needle hub assembly such that the needle hub assembly is attached to a distal end of a syringe, the needle hub assembly comprising a hollow body; a platform; and a needle hub, wherein the needle hub comprising a plurality of needles disposed on the platform arranged in a predefined needle array, wherein the plurality of needles closely spaced, which and are inserted into the predetermined site within the patient for delivering the therapeutic formulation.

Embodiments of the present invention are directed to a medical device for the intracellular delivery of a therapeutic formulation to a predetermined site within a patient' skin and/or a scalp, according to an embodiment of the present invention. The medical device comprising a needle hub assembly such that the needle hub assembly is connected to a distal end of a syringe, the needle hub assembly comprising a hollow body; a platform; and a needle hub, wherein the needle hub comprising a plurality of needles disposed on the platform arranged in a linear pattern sequence, wherein the plurality of needles closely spaced, which are inserted into the predetermined site within the patient for delivering the therapeutic formulation.

Embodiments of the present invention are directed to a medical device for the intracellular delivery of a therapeutic formulation to a predetermined site within a patient, according to an embodiment of the present invention. The medical device comprising a needle hub assembly such that the needle hub assembly is connected to a distal end of a syringe, the needle hub assembly comprising a hollow body; a platform; and a needle hub, wherein the needle hub comprising a plurality of needles disposed on the platform arranged in a concentric circular pattern sequence, wherein the plurality of needles closely spaced and are inserted into the predetermined site within the patient for delivering the therapeutic formulation.

Embodiments of the present invention are directed to significantly reduce time, minimizing pain, and increasing medical formulation absorption efficiency to maximize effectiveness of treatment.

Embodiments in accordance with the present invention provide a medical device for a treatment of skin/tissue such as, skin lifting, reducing skin laxity, skin tightening, stimulate healthier follicles that produces firmer hair, etc.

Embodiments in accordance with the present invention further provide a medical device to inject ‘Platelet Rich Plasma” (PRP) into the epidermis in order to treat fine lines and wrinkles.

Embodiments in accordance with the present invention further provide a medical device having the needle hub assembly that comprises closely packed needles arranged in multiple rows and columns.

Embodiments in accordance with the present invention further provide a medical device having the needle hub assembly that comprises 16 closely packed needles arranged in a concentric circular pattern sequence.

Embodiments in accordance with the present invention further provide a medical device having the needle hub assembly that comprises 16 closely packed needles arranged in a linear pattern sequence.

Embodiments of the present invention may provide a number of advantages depending on its particular configuration. It is an object of the present invention to provide a medical device that may be effective in injecting medical formulations into the skin infusing the epidermis or dermis with Platelet Rich Plasma (PRP) proportional. Furthermore, the disclosed medical device comprises a plurality of needles in a small area that may be beneficial for injecting plasma for skin and scalp injections, infusing, penetrating the PRP into a targeted area only.

These and other advantages will be apparent from the present application of the embodiments described herein. The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

The foregoing and other aspects of the embodiments disclosed herein are best understood from the following detailed description when read in connection with the accompanying drawings. For the purpose of illustrating the embodiments disclosed herein, there is shown in the drawings embodiments that are presently preferred, it being understood, however, that the embodiments disclosed herein are not limited to the specific instrumentalities disclosed. Included in the drawings are the following figures:

FIG. 1 illustrates an exploded perspective view of a medical device, according to an embodiment of the present invention disclosed herein;

FIG. 2 illustrates an assembled side view of the medical device, according to an embodiment of the present invention disclosed herein;

FIG. 3 illustrates dimensions of different components of the medical device, according to an embodiment of the present invention disclosed herein;

FIG. 4 illustrates a side view of a needle hub assembly of the medical device, according to an embodiment of the present invention disclosed herein;

FIG. 5 illustrates a top view of the needle hub assembly of the medical device, according to an embodiment of the present invention disclosed herein;

FIG. 6 illustrates a side perspective view of a needle hub assembly of the medical device, according to an embodiment of the present invention disclosed herein;

FIG. 7 illustrates a bottom view of the needle hub assembly of the medical device, according to an embodiment of the present invention disclosed herein;

FIG. 8 illustrates a perspective view of a needle cap of the needle hub assembly of the medical device, according to an embodiment of the present invention disclosed herein;

FIG. 9 illustrates an exploded side perspective view of a medical device, according to another embodiment of the present invention disclosed herein;

FIG. 10 illustrates an assembled view of the medical device, according to another embodiment of the present invention disclosed herein;

FIGS. 11A-11C illustrate dimensions of different components of the medical device, according to another embodiment of the present invention disclosed herein; and

FIGS. 12A-12D illustrate different views of a needle hub assembly of the medical device, according to another embodiment of the present invention disclosed herein.

While embodiments of the present invention are described herein by way of example using several illustrative drawings, those skilled in the art will recognize the present invention is not limited to the embodiments or drawings described. It should be understood the drawings and the detailed description thereto are not intended to limit the present invention to the particular form disclosed, but to the contrary, the present invention is to cover all modification, equivalents and alternatives falling within the spirit and scope of embodiments of the present invention as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

### DETAILED DESCRIPTION

Embodiments of the present invention will be described below in conjunction with exemplary medical equipment. Embodiments of the present invention are not limited to any particular type of medical equipment. Those skilled in the art will recognize the disclosed techniques may be used in any medical equipment in which it is desirable to inject medication in the layers of skin of a patient.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

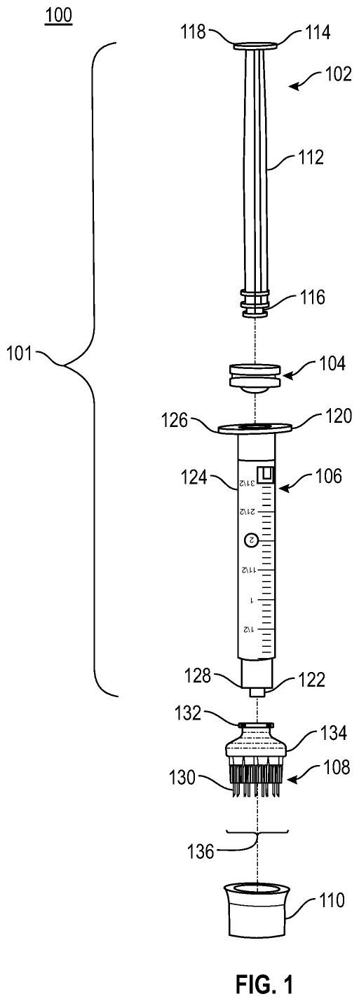

FIG. 1 illustrates an exploded perspective view of a medical device 100, according to an embodiment of the present invention. The medical device 100 comprises a syringe 101. In an embodiment of the present invention, the syringe 101 may be any 3 ml syringe. In another embodiment of the present invention, the syringe 101 may be any 5 ml syringe. Embodiments of the present invention are intended to include or otherwise cover any type of a syringe 101 including known, related art, and/or later developed technologies. The syringe 101 further comprises a plunger 102, a plunger piston 104 and a barrel 106. The medical device 100 further comprises a needle hub assembly 108 and a needle cap 110. In an embodiment of the present invention, the plunger 102 comprises a plunger rod 112, a proximal end 114, and a distal end 116. The proximal end 114 and the distal end 116 may be connected through the plunger rod 112. In an embodiment of the present invention, the plunger rod 112 may be cylindrical in shape. In another embodiment of the present invention, the plunger rod 112 may be of any other shape. In an embodiment of the present invention, the plunger 102 may be made up of, but is not limited to, rigid plastic and/or other material known to a person skilled in the art.

Further, the proximal end 114 of the plunger 102 may include a thumbpress 118, in an embodiment of the present invention. The thumbpress 118 may be configured to push and/or pull the plunger 102 for receiving and/or injecting medical formulations at a predetermined site within a patient. In an embodiment of the present invention, the predetermined site within a patient may be, but not limited to, a skin, a scalp, and so forth. In an embodiment of the present invention, the plunger 102 may be made up of plastic. Embodiments of the present invention are intended to include or otherwise cover any material for the plunger 102, the plunger rod 112, and the thumbpress 118 including known, related art, and/or later developed technologies that may be beneficial to provide a robust structure.

Further, the distal end 116 of the plunger 102 may comprise the plunger piston 104. In an embodiment of the present invention, the plunger piston 104 may be removably attached to the distal end 116 of the plunger 102. The plunger piston 104 may be made up of, but not limited to, an elastomeric material. Embodiments of the present invention are intended to include or otherwise cover any material for the plunger piston 104 including known, related art, and/or later developed technologies. Furthermore, the plunger piston 104 may comprise a small opening such that the opening may be beneficial for the removal of air from the barrel 106 when the medical device 100 is in use.

Further, the barrel 106 may have a proximal end 120, a distal end 122 and a chamber 124, according to an embodiment of the present invention. The plunger 102 of the medical device 100 may be linearly pulled and/or pushed between the proximal end 120 and the distal end 122 within the barrel 106. The plunger piston 104 may be dimensioned to be frictionally slidable along an inner wall of the barrel 106. Further, the chamber 124 between the proximal end 120 and the distal end 122 of the barrel 106 may be used to retain medical formulations and/or other fluids. In an embodiment of the present invention, the chamber 124 may retain liquids such as, but not limited to, blood that is pulled out from a patient, medical formulations to be injected into the body of the patient, and so forth. In an embodiment of the present invention, the medical device 100 may comprise a measuring scale on the outer wall of the chamber 124 between the proximal end 120 and the distal end 122. The measuring scale may be used to measure the amount of the medical formulation in the chamber 124, in an embodiment of the present invention.

In an embodiment of the present invention, when the plunger 102 is pulled towards the proximal end 120 within the barrel 106, the medical formulation may be drawn into the chamber 124 of the barrel 106 of the medical device 100 from a medical container (not shown). In another embodiment of the present invention, when the plunger 102 is pushed from the proximal end 120 towards the distal end 122 within the barrel 106, the medical formulation may be pushed out from the chamber 124 of the barrel 106 of the medical device 100. The barrel 106 may be made up of, but is not limited to, plastic, glass, and so forth, in an embodiment of the present invention. Embodiments of the present invention are intended to include or otherwise cover any material for the barrel 106 including known, related art, and/or later developed technologies that may be beneficial to retain medical formulations. In one embodiment of the present invention, the medical formulation may be in a liquid form, which may be used to treat skin and/or tissue disorders in a patient.

According to an embodiment of the present invention, the proximal end 120 of the barrel 106 may comprise a finger flange 126. In another embodiment of the present invention, the finger flange 126 may be pre-fabricated along any portion of the barrel 106, or may be a separate component that is connected to or affixed to the barrel 106. The finger flange 126 may be configured to provide an interface for holding the medical device 100 while depressing the plunger 102 for injection of the medical formulation in the patient. Embodiments of the present invention are intended to include or otherwise cover any shape, size, and material for the finger flange 126 including known, related art, and/or later developed technologies.

Further, the distal end 122 of the barrel 106 may comprise a tip 128, according to an embodiment of the present invention. The tip 128 may be utilized to attach a needle hub assembly 108 with the barrel 106. In an embodiment of the present invention, the tip 128 may comprise a threaded male part (not shown) to receive the needle hub assembly 108. In another embodiment of the present invention, a luer locking arrangement (not shown) may be used such that the needle hub assembly 108 may be coupled with the barrel 106 just prior to use.

Further, the needle hub assembly 108 may comprise a plurality of needles 130, in an embodiment of the present invention. The needle hub assembly 108 may comprise an opening 132 to pass the medical formulation from the barrel 106 of the syringe 101 to the plurality of needles 130. The plurality of needles 130 may be disposed on a platform 134 of the needle hub assembly 108. In an embodiment of the present invention, the platform 134 of the needle hub assembly 108 may be, but is not limited to, a square platform. In another embodiment of the present invention, the platform 134 of the needle hub assembly 108 may be a circular platform. Embodiments of the present invention are intended to include or otherwise cover any shape for the platform 134 including known, related art, and/or later developed technologies. According to an embodiment of the present invention, the platform 134 may be but is not limited to, a flat platform.

In an embodiment of the present invention, each of the plurality of needles 130 may comprise an outlet (not shown) at a proximal end of the needle to receive the therapeutic formulation from the barrel 106. Further, each of the plurality of needles 130 comprising an opening at a distal end of the needle to deliver the therapeutic formulation at the predetermined site of a patients' skin or scalp. The plurality of needles 130 may be arranged in a multiple row and multiple column needle array 136 arranged on the platform 134. In an embodiment of the present invention, the plurality of needles 130 are arranged in a concentric circular pattern sequence. In another embodiment of the present invention, the plurality of needles 130 are arranged in a linear pattern sequence.

In one embodiment of the present invention, the needle array 136 may include 16 needles. In another embodiment, the needle array 136 may include any number of needles that may be beneficial, for example, to inject medical formulations such as, but not limited to, “Platelet Rich Plasma” in the epidermis of a patient simultaneously through the plurality of needles 130. In an embodiment of the present invention, the plurality of needles 130 may be placed in a 4×4 needle array on the platform 134. In a further embodiment of the present invention, the plurality of needles 130 may be placed in a 2×8 needle array on the platform 134. Further, the needle array 136 may be a disposable needle array, in one embodiment of the present invention. In another embodiment of the present invention, the needle array 136 may be a re-usable needle array.

Further, the needle cap 110 may be provided to seal the medical device 100 filled with the medical formulation or other liquid before storing and/or moving the medical device 100. In an embodiment of the present invention, the needle cap 110 may be made up of, but is not limited to, rigid plastic or other material known to person skilled in the art. According to an embodiment of the present invention, the shape of the needle cap 110 may be, but is not limited to a square or a circle. According to an embodiment of the present invention, the shape of the needle cap 110 may be, but not limited to, as per the shape of the needle hub assembly 108.

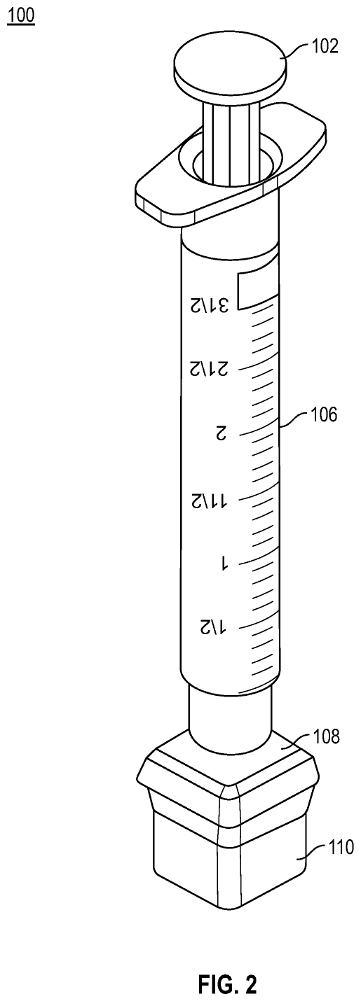

FIG. 2 illustrates an assembled side perspective view of the medical device 100, according to an embodiment of the present invention. The needle cap 110 may be removably attached to the needle hub assembly 108. The needle cap 110 may completely cover the needle hub assembly 108 in a secured position to protect the tips of the plurality of needles 130 (not shown) from contamination.

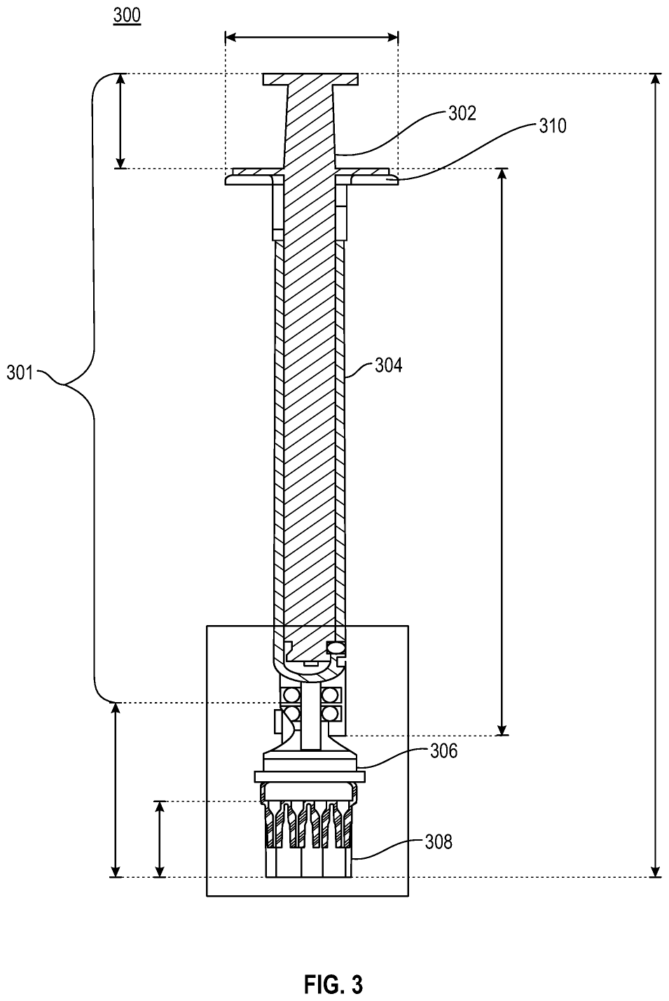

FIG. 3 illustrates dimensions of different components of the medical device 300, according to an embodiment of the present invention. The medical device 300 comprises a syringe 301a needle hub assembly 306 and a needle cap 308. The syringe 301 further comprises a plunger 302 and a barrel 304. The medical device 300 may comprise a total length of 4.351 inches, in an embodiment of the present invention. The length of the barrel 304 may be 3.057 inches. Further, the length of the needle hub assembly 306 may be 0.940 inches. In an embodiment of the present invention, the length of the needle hub assembly 306 may be in a range from 0.920 inches to 1.0 inches. The length of the plunger 302 may be approximately 3.411 inches. According to an embodiment of the present invention, the proximal end of the barrel 304 may comprise a finger flange 310. The finger flange 310 may be pre-fabricated along any portion of the barrel 304, or may be removably connected to or affixed to the barrel 304. The width of the finger flange 310 may be 0.961 inches, in an embodiment of the present invention. Further, the length of the portion of the plunger 302 that may be left outside the barrel 304 when the plunger 302 is fully depressed into the barrel 304 may be 0.503 inches. According to an embodiment of the present invention, the length of each of the plurality of needles 308 may be 0.75 inches. In an embodiment of the present invention, the length of each of the plurality of needles 308 may be in a range from 0.65 inches to 0.85 inches. Further, each of the plurality of needles 308 may be a 30 gauge needle. Embodiments of the present invention are intended to include or otherwise cover any type of the plurality of needles 308 including known, related art, and/or later developed technologies.

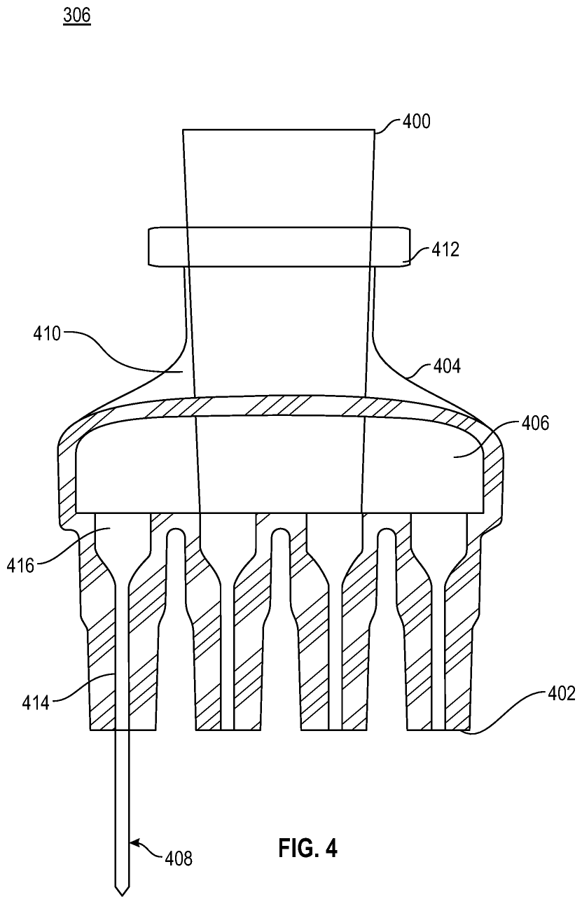

FIG. 4 illustrates a side view of the needle hub assembly 306 of the medical device 300, according to an embodiment of the present invention. The needle hub assembly 306 may comprise a proximal end 400, a distal end 402, a hollow body 404, a platform 406, a plurality of needles 408, a needle hub 410 and a lock 412. The hollow body 404 of the needle hub assembly 306 may be made up of, but not limited to, plastic. Embodiments of the present invention are intended to include or otherwise cover any material for the hollow body 404 including known, related art, and/or later developed technologies. In an embodiment of the present invention, the hollow body 404 may be a square-shaped plastic body formed by injection molding. In another embodiment of the present invention, the hollow body 404 may be a square plastic body formed by any other molding technique, including known, related art, and/or later developed technologies. Further, the hollow body 404 may comprise the platform 406 that may be utilized to hold a plurality of needles 408. The plurality of needles 408 may be made up of, but is not limited to, stainless steel. Embodiments of the present invention are intended to include or otherwise cover any material for the plurality of needles 408 including known, related art, and/or later developed technologies. According to an embodiment of the present invention, the needles 408 may be hollow needles.

Further, the platform 406 may comprises a plurality of grooves 414 for accepting a plurality of needles 408. According to an embodiment of the present invention, the plurality of grooves 414 may be arranged in a 4×4 needle array sequence, and/or a linear pattern sequence. Further, the plurality of grooves 414 may be arranged in a 2×8 needle array sequence, and/or a linear pattern sequence, according to another embodiment of the present invention. According to an exemplary embodiment of the present invention, the plurality of grooves 414 may be arranged in a concentric circular pattern sequence. Furthermore, the grooves 414 may comprise an outlet 416. The outlet 416 may communicate with the barrel 106 of the syringe 101 to receive the medical formulation from the barrel 106 and transmit the medical formulation at a predetermined site of a patient from the opening of each of the plurality of needles 408 through the outlet 416.

Further, the needle hub assembly 306 comprises the lock 412 at the proximal end 400 of the needle hub assembly 306. In one embodiment of the present invention, the lock 412 may be, but is not limited to, a luer lock. In another embodiment of the present invention, the lock 412 may be any other lock that may be beneficial to attach the syringe 301 (not shown) of the medical device 100 to the needle hub assembly 306 of the medical device 100. More specifically, the lock 412 may attach the needle hub assembly 306 with the tip 128 (not shown) of the barrel 106 of the syringe 301 (not shown).

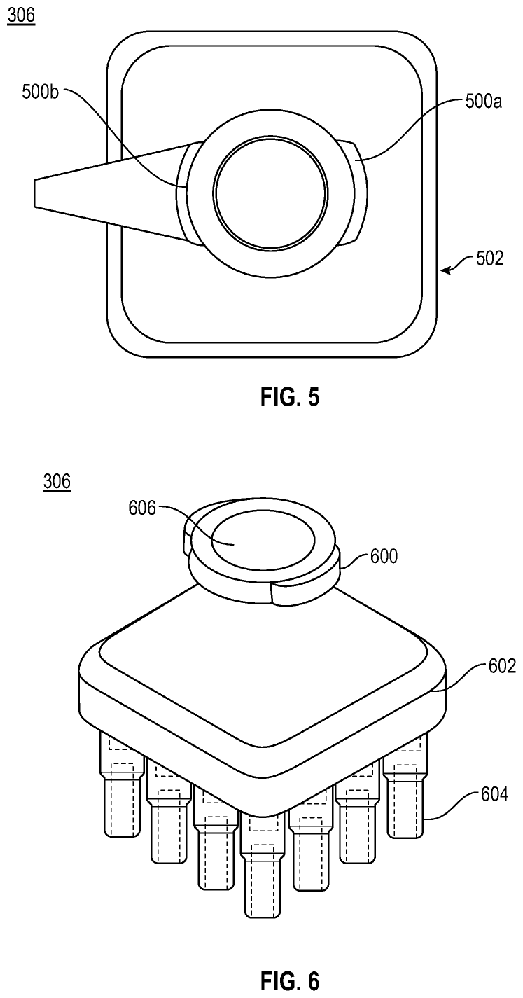

FIG. 5 illustrates a top view of the needle hub assembly 306 of the medical device 300, according to an embodiment of the present invention. The top view illustrates finger flanges 500a and 500b (hereinafter referred to as “finger flange 500”) and a square platform 502 of the needle hub assembly 306.

FIG. 6 illustrates a side perspective view of the needle hub assembly 306 of the medical device 300, according to an embodiment of the present invention. The needle hub assembly 306 may comprise a finger flange 600, a square platform 602, a plurality of grooves 604 and an opening 606. The finger flange 600 may be configured to provide an interface for holding the medical device 100. Further, the position, shape, number, and materials for such components may vary, as would be readily appreciated by a skilled art, to meet any number of desired characteristics. Further, the square platform 602 may be configured to hold the plurality of needles (not shown). The plurality of needles may be pre-assembled into the plurality of grooves 604 of the medical device 300. Further, the needle hub assembly 306 may have the opening 606 to pass the medical formulations to the barrel 106 (not shown) from an object such as, but not limited to, a bottle, skin, etc. and vice versa.

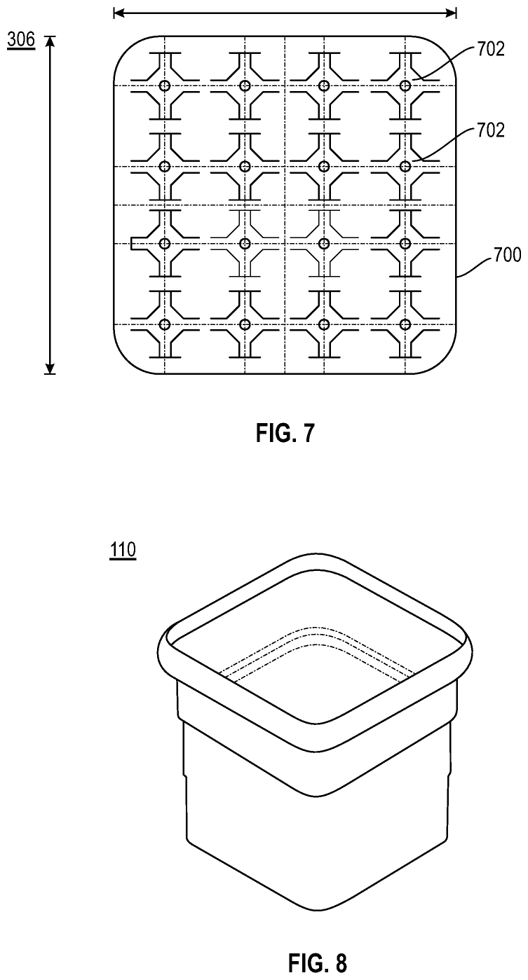

FIG. 7 illustrates a bottom view of the needle hub assembly 306 of the medical device 300, according to an embodiment of the present invention disclosed herein. In one embodiment of the present invention, the needle hub assembly 306 may be made up of, but is not limited to, clear polypropylene and/or any such material known to a person skilled in the art. The needle hub assembly 306 may include a platform 700 to hold a plurality of needles (not shown) in the grooves 702 in a fixed position. In an embodiment of the present invention, the platform 700 may be a square shaped platform. In an embodiment of the present invention, a length of each side of the platform 700 may be 0.5 inches. In another embodiment of the present invention, the length of each side of the platform 700 may be in a range from 0.4 to 0.9 inches. In another embodiment of the present invention, the platform 700 may be circular shaped platform. The needles may be arranged in multiple rows and columns in order to cover a larger area of a skin to inject the medical formulation such as platelet rich plasma

(PRP), hyaluronic acid and/or anti-inflammatory steroid. The needles may be arranged in a 4×4 array on the platform 700, in one embodiment. The platform 700 may be a 16 needle platform that may be disposable and screwed onto the medical device 100 to inject, for example, platelet rich plasma (PRP) in square footprints into the epidermis and dermis of the face, skin, and or scalp. The grooves 702 may be 0.0625 inch apart, in one embodiment of the present invention. In another embodiment of the present invention, a distance between the grooves 702 may be in a range of 0.055 inches to 0.075 inches. The needles may be closely placed to insure that the target area is properly infiltrated for best results. In one embodiment of the present invention, short 31 gauge needles may be used to infuse small areas such as crow's feet with PRP to regenerate healthy skin growth. In another embodiment of the present invention, slightly longer 30 gauge needle heads may be used to infiltrate underlying dermis to regenerate healthy deeper tissue growth. The medical device 100 with 16 needles head of 30 gauge needles may be used to inject the scalp to revitalize weak and dormant hair follicles to stimulate healthy hair growth.

FIG. 8 is a diagram illustrating the protective needle cap 110 of the needle hub assembly 108 of the multi-needle medical device 100, according to embodiments disclosed herein. In one embodiment of the present invention, the needle cap 110 may be made up of, but is not restricted to, brown polypropylene, or any other material that may be feasible to protect the needles from contamination.

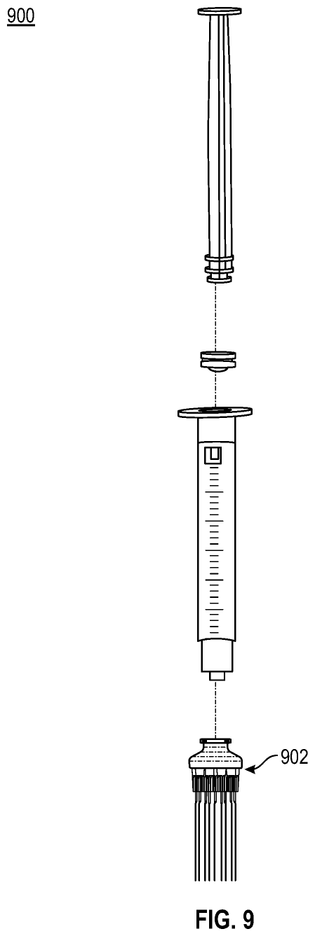

FIG. 9 illustrates an exploded side perspective view of a medical device 900, according to another embodiment of the present invention. According to an exemplary embodiment of the present invention the medical device 900 may comprise a needle hub assembly 902 with a circular platform.

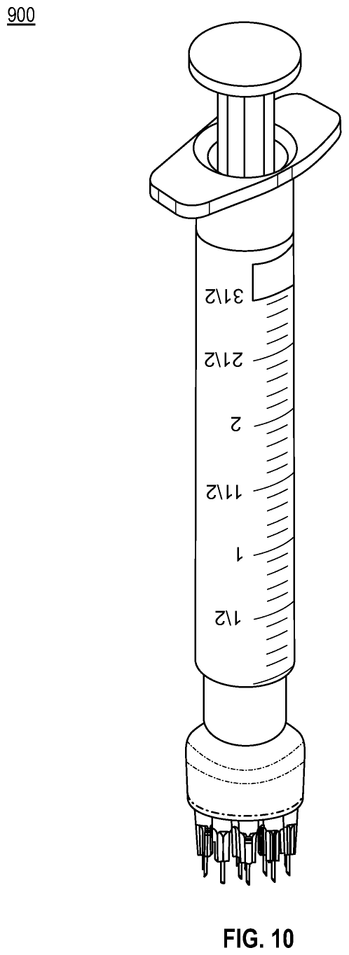

FIG. 10 illustrates an assembled view of the medical device 900, according to an embodiment of the present invention disclosed herein.

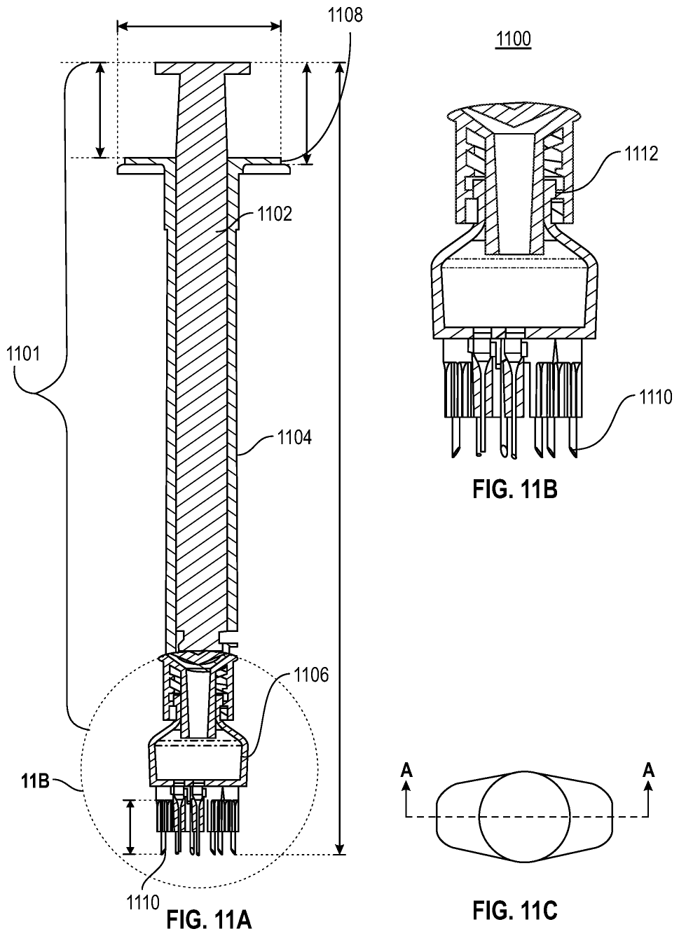

FIGS. 11A-11C illustrate dimensions of different components of a medical device 1100, according to an embodiment of the present invention. The medical device 1100 comprises a syringe 1101 and a needle hub assembly 1106. Further, the syringe 1101 comprises a plunger 1102 and a barrel 1104. The medical device 1100 may comprise a total length of 4.294 inches, in an embodiment of the present invention. The total length of the barrel 1104 and the portion of the plunger 1102 outside the barrel 1104 when the plunger 1102 is fully depressed may be 3.560 inches. Further, the length of the needle hub assembly 1106 as shown in the FIG. 11B, may be 0.874 inches. Further, the length of the portion of the plunger 1102 outside the barrel 1104 when the plunger 1102 is fully depressed into the barrel 1104 may be 0.503 inches. According to an embodiment of the present invention, a proximal end of the barrel 1104 may comprise a finger flange 1108. The finger flange 1108 may be pre-fabricated along any portion of the barrel 1104, or may be a removably connected to or affixed to the barrel 1104. The width of the finger flange 1108 may be 0.960 inches, in an embodiment of the present invention. Further, as shown in the FIG. 11B, a length of the needles 1110 may be 0.75 inches and the height of the needles 1110 protruding out of the grooves of the needle hub assembly 1106 may be 0.125 inches. Further, each needle 1110 may be a 30 gauge needle. Embodiments of the present invention are intended to include or otherwise cover any type of needles 1110 including known, related art, and/or later developed technologies. Furthermore, the length of the luer lock 1112 may be 0.140 inches, in an embodiment of the present invention. FIG. 11C illustrates a top view of the finger flange 1108 according to an embodiment of the present invention.

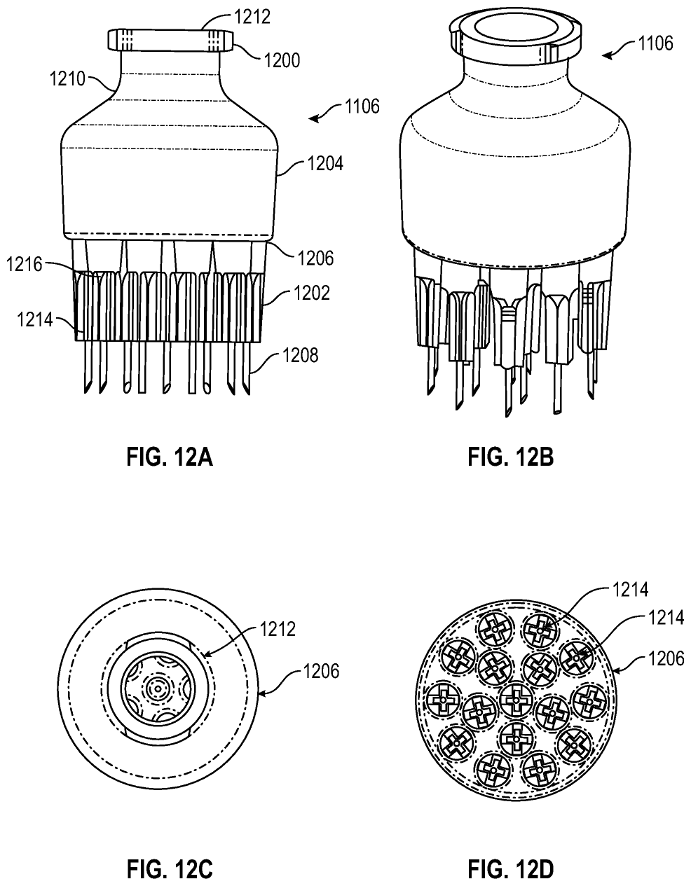

FIGS. 12A-12D illustrate different views of the needle hub assembly 1106 of the medical device 1100, according to embodiment of the present invention. FIG. 12A illustrates a side view of the needle hub assembly 1106 of the medical device 1100, according to an exemplary embodiment of the present invention. The needle hub assembly 1106 may comprise a proximal end 1200, a distal end 1202, a hollow body 1204, a platform 1206, a plurality of needles 1208, a needle hub 1210 and a lock 1212. The hollow body 1204 of the needle hub assembly 1206 may be made up of, but not limited to, plastic. Embodiments of the present invention are intended to include or otherwise cover any material for the hollow body 1204 including known, related art, and/or later developed technologies. In an embodiment of the present invention, the hollow body 1204 may be a circular plastic body formed by injection molding. In another embodiment of the present invention, the hollow body 1204 may be a circular plastic body formed by any other molding technique, including known, related art, and/or later developed technologies. Further, the hollow body 1204 may comprise the platform 1206 that may be utilized to hold the plurality of needles 1208. The plurality of needles 1208 may be made up of, but is not limited to, stainless steel. Embodiments of the present invention are intended to include or otherwise cover any material for the plurality of needles 1208 including known, related art, and/or later developed technologies. According to an embodiment of the present invention, the needles 1208 may be hollow needles.

Further, the platform 1206 may comprises a plurality of grooves 1214 for accepting the plurality of needles 1208. According to an embodiment of the present invention, the plurality of grooves 1214 may be arranged in a concentric circular pattern sequence. Furthermore, each of the plurality of grooves 1214 may comprise an outlet 1216. The outlet 1216 may communicate with the barrel 1104 of the medical device 1100 to receive the medical formulation from the barrel 1104 and transmit the medical formulation at a predetermined site of a patient from the opening of each of the plurality of needles 1208 through the outlet 1216.

Further, the needle hub assembly 1106 comprises the lock 1212 at the proximal end 1200 of the needle hub assembly 1106. In one embodiment of the present invention, the lock 1212 may be, but is not limited to, a luer lock. In another embodiment of the present invention, the lock 1212 may be any other lock that may be beneficial to attach the syringe 1101 of the medical device 1100 to the needle hub assembly 1106 of the medical device 1100. More specifically, the lock 1212 may attach the needle hub assembly 1106 with the tip (not shown) of the barrel 1104 of the syringe 1101.

FIG. 12B illustrates a side perspective view of the needle hub assembly 1106 of the medical device 1100, according to an embodiment of the present invention.

FIG. 12C illustrates a top view of the needle hub assembly 1106 of the medical device 1100, according to an embodiment of the present invention. The top view illustrates the lock 1212 and the platform 1206 of the needle hub assembly 1106, according to an embodiment of the present invention.

FIG. 12D illustrates a bottom view of the needle hub assembly 1106 of the medical device 1100, according to an embodiment of the present invention disclosed herein. In one embodiment of the present invention, the needle hub assembly 1106 may be made up of, but is not limited to, clear polypropylene and/or any such material known to a person skilled in the art. The needle hub assembly 1106 may include a platform 1206 to hold a plurality of needles (not shown) in the grooves 1214 in a fixed position. In an embodiment of the present invention, the platform 1206 may be a circular shaped platform. Further, the platform 1206 may have a diameter of 0.5 inches. In an embodiment of the present invention, the platform 1206 may have a diameter in a range from 0.3 inches to 0.8 inches. The needles may be arranged in concentric circular pattern sequence in order to cover a larger area of a skin to inject the medical formulation such as platelet rich plasma (PRP), hyaluronic acid and/or anti-inflammatory steroid. The platform 1206 may be a 16 needle platform that may be disposable and screwed onto the medical device 1100 to inject, for example, platelet rich plasma (PRP) into the epidermis and dermis of the skin and/or scalp. In an embodiment of the present invention, the grooves 1214 may be arranges in a circular pattern as 9 grooves 1214 on an outer layer on the platform 1206, 5 grooves 1214 in a middle layer on the platform 1206 and 1 groove 1214 in the center of the platform 1206. In another embodiment of the present invention, the groves 1214 may be in another other sequence including known or later developed technologies that may be beneficial to arrange closely spaced needles. The needles may be closely placed to insure that the target area is properly infiltrated for best results. In one embodiment of the present invention, short 31 gauge needles may be used. In another embodiment of the present invention, 0.75 inches long 30 gauge needles may be used to infiltrate underlying dermis to regenerate healthy deeper tissue growth. The medical device 1100 with 16 needles that may be 0.75 inches long and 30 gauge thick may be used to inject the scalp to revitalize weak and dormant hair follicles to stimulate healthy hair growth.

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

The exemplary embodiments of this present invention have been described in relation to medical equipment. However, to avoid unnecessarily obscuring the present invention, the preceding description omits a number of known structures and devices. This omission is not to be construed as a limitation of the scope of the present invention. Specific details are set forth by use of the embodiments to provide an understanding of the present invention. It should however be appreciated that the present invention may be practiced in a variety of ways beyond the specific embodiments set forth herein.

A number of variations and modifications of the present invention can be used. It would be possible to provide for some features of the present invention without providing others.

The present invention, in various embodiments, configurations, and aspects, includes components, methods, processes, systems and/or apparatus substantially as depicted and described herein, including various embodiments, sub-combinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and/or reducing cost of implementation.

The foregoing discussion of the present invention has been presented for purposes of illustration and description. It is not intended to limit the present invention to the form or forms disclosed herein. In the foregoing Detailed Description, for example, various features of the present invention are grouped together in one or more embodiments, configurations, or aspects for the purpose of streamlining the disclosure. The features of the embodiments, configurations, or aspects may be combined in alternate embodiments, configurations, or aspects other than those discussed above. This method of disclosure is not to be interpreted as reflecting an intention the present invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate embodiment of the present invention.

Moreover, though the description of the present invention has included description of one or more embodiments, configurations, or aspects and certain variations and modifications, other variations, combinations, and modifications are within the scope of the present invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A medical device for the intracellular delivery of a therapeutic formulation to a predetermined site within a patient, the medical device comprising:
a needle hub assembly such that the needle hub assembly is attached to a distal end of a syringe, the needle hub assembly comprising:
a hollow body;
a platform; and
a needle hub, wherein the needle hub comprising a plurality of needles disposed on the platform arranged in a predefined needle array, wherein the plurality of needles closely spaced, which are inserted into the predetermined site within the patient for delivering the therapeutic formulation.

2. The device of claim 1, further comprising a needle cap removeable attached to the needle hub assembly in order to seal the needle hub assembly.

3. The device of claim 1, wherein the syringe is a 3 ml disposable syringe.

4. The device of claim 1, wherein each of the plurality of needles comprising an outlet at a proximal end of the needle to receive the therapeutic formulation from a barrel.

5. The device of claim 1, wherein each of the plurality of needles comprising an opening at a distal end of the needle to deliver the therapeutic formulation into a skin of the patient.

6. The device of claim 1, wherein each of the plurality of needles comprising an opening at a distal end of the needle to deliver the therapeutic formulation into a scalp of the patient.

7. The device of claim 1, wherein a shape of the needle hub assembly comprises one of a square, a circle, and a rectangle.

8. The device of claim 1, wherein the platform of the needle hub assembly is flat.

9. The device of claim 1, wherein the plurality of needles are arranged in a concentric circular pattern sequence.

10. The device of claim 1, wherein the plurality of needles are arranged in a linear pattern sequence.

11. The device of claim 1, wherein a dimension of each of the plurality of needles is 30 gauge×0.75 inches.

12. The device of claim 1, further comprising a lock to assemble the syringe and the needle hub assembly.

13. A medical device for the intracellular delivery of a therapeutic formulation to a predetermined site within a patient, the medical device comprising:
a needle hub assembly such that the needle hub assembly is attached to a distal end of a syringe, the needle hub assembly comprising;
a hollow body;
a platform; and
a needle hub, wherein the needle hub comprising a plurality of needles disposed on the platform arranged in a linear pattern sequence, wherein the plurality of needles closely spaced, which are inserted into the predetermined site within the patient for delivering the therapeutic formulation.

14. The device of claim 13, further comprising a needle cap removeable attached to the needle hub assembly in order to seal the needle hub assembly.

15. The device of claim 13, wherein the syringe is a 3 ml disposable syringe.

16. The device of claim 13, wherein a length of each side of the platform is 0.5 inches.

17. The device of claim 13, wherein each of the plurality of needles comprising an outlet at a proximal end of the needle to receive the therapeutic formulation from the syringe.

18. The device of claim 13, wherein each of the plurality of needles comprising an opening at a distal end of the needle to deliver the therapeutic formulation at the predetermined site.

19. The device of claim 13, wherein the plurality of needles are arranged in a 4×4 needle array.

20. The device of claim 13, wherein the plurality of needles are arranged in a 2×8 needle array.

21. The device of claim 13, wherein a dimension of each of the plurality of needles is 30 gauge×0.75 inches.

22. The device of claim 13, further comprising a lock to assemble the syringe and the needle hub assembly.

23. A medical device for the intracellular delivery of a therapeutic formulation to a predetermined site within a patient's skin and/or scalp, the medical device comprising:
a needle hub assembly such that the needle hub assembly is attached to a distal end of a syringe, the needle hub assembly comprising:
a hollow body;
a platform; and
a needle hub, wherein the needle hub comprising a plurality of needles disposed on the platform arranged in a concentric circular pattern sequence, wherein the plurality of needles are closely spaced, which are inserted into the predetermined site within the patient's skin and/or scalp for delivering the therapeutic formulation.

24. The device of claim 23, further comprising a needle cap removeable attached to the needle hub assembly in order to seal the needle hub assembly.

25. The device of claim 23, wherein the syringe is a 3 ml disposable syringe.

26. The device of claim 23, wherein a diameter of the platform of the needle hub assembly is 0.5 inches.

27. The device of claim 23, wherein each of the plurality of needles comprising an outlet at a proximal end of the needle to receive the therapeutic formulation from the barrel.

28. The device of claim 23, wherein a dimension of each of the plurality of needles is 30 gauge×0.75 inches.

29. The device of claim 23, further comprising a lock to assemble the syringe and the needle hub assembly.

30. The device of claim 23, wherein the therapeutic formulation is delivered into a skin infusing an epidermis, or dermis of the patient.
